(19) United States
(12) Patent Application Publication
(10) Pub. No.: US 2017/0252002 A1
(43) Pub. Date: Sep. 7, 2017

(54) ULTRASONIC DIAGNOSTIC APPARATUS AND ULTRASONIC DIAGNOSIS SUPPORT APPARATUS

(71) Applicant: TOSHIBA MEDICAL SYSTEMS CORPORATION, Otawara-shi (JP)

(72) Inventors: Yoshitaka MINE, Nasushiobara (JP); Kazutoshi SADAMITSU, Otawara (JP); Masami TAKAHASHI, Nasushiobara (JP); Masatoshi NISHINO, Otawara (JP); Norihisa KIKUCHI, Otawara (JP); Naoyuki NAKAZAWA, Otawara (JP); Atsushi NAKAI, Nasushiobara (JP); Jiro HIGUCHI, Otawara (JP); Yutaka KOBAYASHI, Nasushiobara (JP); Cong YAO, Otawara-shi (JP); Kazuo TEZUKA, Nasushiobara (JP); Naoki YONEYAMA, Yaita (JP); Atsushi SUMI, Otawara (JP)

(73) Assignee: TOSHIBA MEDICAL SYSTEMS CORPORATION, Otawara-shi (JP)

(21) Appl. No.: 15/450,859

(22) Filed: Mar. 6, 2017

(30) Foreign Application Priority Data
Mar. 7, 2016 (JP) 2016-043828
Feb. 8, 2017 (JP) 2017-021034

(51) Int. Cl. A61B 8/00; A61B 6/03; A61B 5/00; A61B 5/0205; A61B 5/0402; A61B 8/14; A61B 8/08

(52) U.S. Cl. CPC A61B 8/4218; A61B 8/145; A61B 8/4466; A61B 8/4254; A61B 8/4263; A61B 6/032; A61B 8/5261; A61B 5/0205; A61B 5/0402; A61B 8/543; A61B 5/0077; A61B 8/467; A61B 8/461; A61B 5/055

(57) ABSTRACT

In one embodiment, an ultrasonic diagnostic apparatus includes an ultrasonic probe; a robot arm configured to support the ultrasonic probe and move the ultrasonic probe along a body surface of an object; memory circuitry configured to store trace instruction information used by the robot arm for moving the ultrasonic probe; and control circuitry configured to drive the robot arm in such a manner that the robot arm moves the ultrasonic probe according to the trace instruction information.

### CROSS-REFERENCE TO RELATED APPLICATIONS

This application is based upon and claims the benefit of priority from Japanese Patent Application No. 2016-043828, filed on Mar. 7, 2016 and Japanese Patent Application No. 2017-021034 filed on Feb. 8, 2017, the entire contents of each of which are incorporated herein by reference.

### FIELD

Embodiments described herein relate generally to an ultrasonic diagnostic apparatus and an ultrasonic diagnosis support apparatus.

### BACKGROUND

An ultrasonic diagnostic apparatus is configured to non-invasively acquire information inside an object by transmitting an ultrasonic pulse and/or an ultrasonic continuous wave generated by a transducer included in an ultrasonic probe to an object's body and converting a reflected ultrasonic wave caused by difference in acoustic impedance between respective tissues in the object into an electric signal. In a medical examination using an ultrasonic diagnostic apparatus, various types of moving image data and/or real-time image data can be easily acquired by scanning an object such that an ultrasonic probe is brought into contact with a body surface of the object. Thus, an ultrasonic diagnostic apparatus is widely used for morphological diagnosis and functional diagnosis of an organ.

Additionally, a three-dimensional ultrasonic diagnostic apparatus is known, which is equipped with a one-dimensional array probe configured to mechanically swing or rotate, or equipped with a two-dimensional array probe, for acquiring three-dimensional image data. Further, a four-dimensional ultrasonic diagnostic apparatus configured to time-sequentially acquire three-dimensional image data substantially on a real-time basis is also known.

Moreover, an ultrasonic diagnostic apparatus equipped with a robot arm configured to hold and move an ultrasonic probe by programing a body-surface scanning procedure of a skilled operator is proposed as an attempt to shorten an examination time.

Meanwhile, it is said that objectivity of diagnosis using an ultrasonic diagnostic apparatus is low compared with objectivity of diagnosis using a computed tomography (CT) apparatus or a magnetic resonance imaging (MRI) apparatus. One of the reasons is that acquisition of ultrasonic images greatly depends on skills of an operator such as a medical doctor or an ultrasonic technician.

For instance, scanning directions of each organ differ depending on its clinical case or symptom, and thus acquired images differ significantly for each operator even in the case of examining the same organ. Since image quality of an ultrasonic image is influenced by factors such as gas, bones, and artifact, it is required to set the optimum position and the optimum angle of a probe according to examination purpose of an object and to scan the object by moving the probe along the optimum path. However, this leads to one of the reasons that image quality of ultrasonic images greatly depends on skills of an operator. Additionally, since only the ultrasonic images selected by an operator are stored, to objectively observe a clinical case only from stored ultrasonic images is difficult in some cases for a doctor who is not involved with the probe operation of the stored ultrasonic images. Further, it is difficult for some hospitals to stably secure sufficiently skilled doctors and/or ultrasonic technicians.

Since a probe is manually moved on a body surface in an ultrasonic scan, it is difficult even for a skilled doctor or an ultrasonic technician to move the probe at a constant speed through the entire scan time. In other words, it is difficult even for a skilled doctor or an ultrasonic technician to acquire cross-sectional ultrasonic images at constant intervals. Additionally, in a routine examination of examining the entirety of plural organs like a health checkup, whether all the target organs are completely scanned or not is judged by an operator's subjectivity and cannot be objectively confirmed.

For this reason, an ultrasonic diagnostic apparatus and an ultrasonic diagnosis support apparatus in each of which the above-described various problems attributable to manual probe movement are resolved have been desired.

### BRIEF DESCRIPTION OF THE DRAWINGS

In the accompanying drawings:

FIG. 1 is a block diagram illustrating basic configuration of the ultrasonic diagnostic apparatus of the present embodiment;

FIG. 2 is a block diagram illustrating configuration of the ultrasonic diagnostic apparatus according to the first modification of the present embodiment;

FIG. 3 is a block diagram illustrating configuration of the ultrasonic diagnostic apparatus according to the second modification of the present embodiment;

FIG. 4 is a block diagram illustrating configuration of the ultrasonic diagnostic apparatus according to the third modification of the present embodiment;

FIG. 5 is a block diagram illustrating more detailed configuration of the ultrasonic diagnostic apparatus of the present embodiment;

FIG. 6 is a block diagram illustrating more detailed configuration of the ultrasonic diagnostic apparatus according to the first modification of the present embodiment;

FIG. 7 is a block diagram illustrating more detailed configuration of the ultrasonic diagnostic apparatus according to the second modification of the present embodiment;

FIG. 8 is a block diagram illustrating more detailed configuration of the ultrasonic diagnostic apparatus according to the third modification of the present embodiment;

FIG. 9 is a flowchart illustrating the first case of a phase in which reference trace information is generated;

FIG. 10 is a flowchart illustrating the second case of the phase in which reference trace information is generated;

FIG. 11 is a schematic diagram illustrating a case of reference trace information and a biological reference position;

FIG. 12 is a flowchart illustrating processing of the second phase in which trace instruction information is generated by correcting or editing reference trace information;

FIG. 13 is a schematic diagram illustrating the first case of generating trace instruction information by correcting reference trace information;

FIG. 14 is a schematic diagram illustrating the second case of generating trace instruction information by correcting reference trace information;

FIG. 15 is a schematic diagram illustrating the third case of generating trace instruction information by correcting reference trace information;

FIG. 16 is a schematic diagram illustrating the fourth case of generating trace instruction information by correcting reference trace information;

FIG. 17 is a schematic diagnostic image illustrating how trace instruction information is generated by correcting reference trace information based on a CT image and/or an MRI image;

FIG. 18 is a schematic diagram illustrating how the optimized trace instruction information is generated by performing optimization processing on plural sets of reference trace information;

FIG. 19 is a flowchart illustrating the third phase in which the robot arm is driven according to trace instruction information; and

FIG. 20 is a block diagram illustrating general configuration of the ultrasonic diagnosis support apparatus of the present embodiment.

### DETAILED DESCRIPTION

Hereinafter, embodiments of ultrasonic diagnostic apparatuses and ultrasonic diagnosis support apparatuses will be described with reference to the accompanying drawings.

In one embodiment, an ultrasonic diagnostic apparatus includes an ultrasonic probe; a robot arm configured to support the ultrasonic probe and move the ultrasonic probe along a body surface of an object; memory circuitry configured to store trace instruction information used by the robot arm for moving the ultrasonic probe; and control circuitry configured to drive the robot arm in such a manner that the robot arm moves the ultrasonic probe according to the trace instruction information.

(General Configuration)

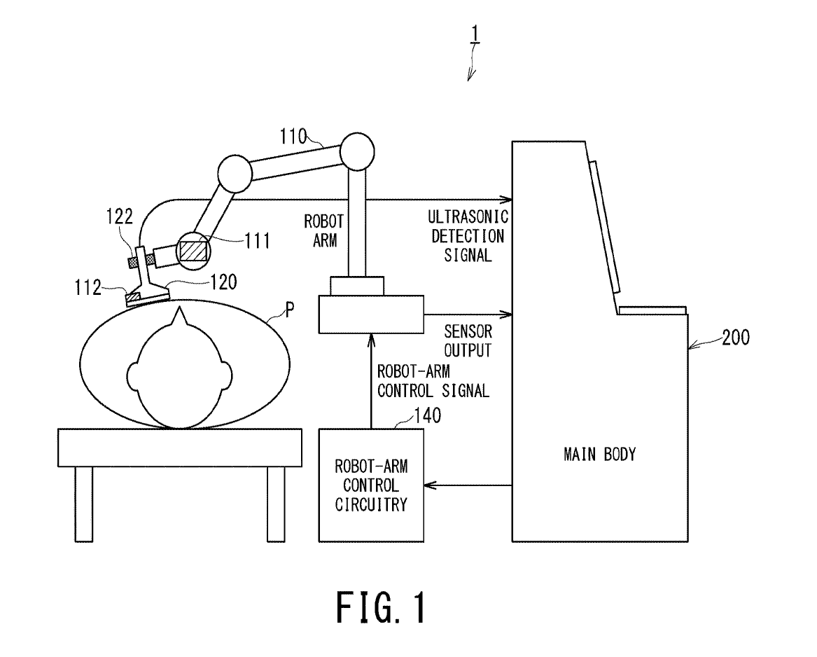

FIG. 1 is a block diagram illustrating basic configuration of the ultrasonic diagnostic apparatus 1 of the present embodiment. The ultrasonic diagnostic apparatus 1 includes at least a main body of the apparatus (hereinafter, simply referred to as the main body 200), an ultrasonic probe 120, a robot arm 110, and robot-arm control circuitry 140.

The robot arm 110 holds (i.e., supports) the ultrasonic probe 120 by, e.g., its end, and can move the ultrasonic probe 120 with six degrees of freedom according to a control signal inputted from the robot-arm control circuitry 140. To be able to move the ultrasonic probe 120 with six degrees of freedom means, e.g., to be able to move it at arbitrary combination of six components including three translation direction components (X, Y, Z) and three rotational direction components (θx, θy, θz). The above-described three translation direction components (X, Y, Z) correspond to an X-axis direction, a Y-axis direction, and a Z-axis direction being perpendicular to each other. The above-described three rotational directions correspond to rotation about the X-axis, rotation about the Y-axis, and rotation about the Z-axis. In other words, the robot arm 110 can locate the ultrasonic probe 120 at a desired position and at a desired orientation in three-dimensional space, and can move the ultrasonic probe 120 along a desired path at a desired velocity.

The robot arm 110 is provided with an arm sensor 111, and detects motions of respective parts of the robot arm 110 by the arm sensor 111. At least a position sensor is included in the arm sensor 111 of the robot arm 110, and the robot arm 110 detects the above-described six components by using this position sensor. Additionally, a velocity sensor may be included in the arm sensor 111 of the robot arm 110 in addition to the position sensor. Further, an acceleration sensor may be included in the arm sensor 111 of the robot arm 110 in addition to the position sensor and the velocity sensor.

Moreover, the robot arm 110 preferably includes a pressure sensor as the arm sensor 111. Biological contact pressure of the ultrasonic probe 120 is transmitted to the robot arm 110 via an ultrasonic probe adapter 122, and is detected by the pressure sensor included in the robot arm 110.

Although FIG. 1 illustrates a case where respective sensors of the arm sensor 111 are disposed at the joint of the end part of the robot arm 110, positions of respective sensors of the arm sensor 111 is not limited to one position. When the robot arm 110 is equipped with plural joints as illustrated in FIG. 1, the arm sensor 111 may be disposed at a position other than a joint position, and the plural sensors of the arm sensor 111 may be dispersed so as to be disposed at respective joints.

Additionally or alternatively to the above-described arm sensor 111, one or more probe sensor 112 such as a pressure sensor, a position sensor, a velocity sensor, an acceleration sensor, and/or a gyroscope sensor may be mounted on the ultrasonic probe 120.

Respective detection signals of the position sensor and the pressure sensor and/or respective detection signals of the velocity sensor and the acceleration sensor are used for feedback control performed by the robot-arm control circuitry 140. As described below, the robot arm 110 is driven by the robot-arm control circuitry 140 according to trace instruction information. The trace instruction information is information defining a position, orientation, a moving path, moving velocity of the ultrasonic probe 120 and biological contact pressure of the ultrasonic probe 120. The moving path is basically defined by three-dimensional coordinate space (i.e., robot coordinate system) inside which the robot arm moves. Further, in order to associate the position of the ultrasonic probe 120 with an observation target such as a biological organ, association information between a biological coordinate system to be set with respect to a living body and the robot coordinate system may be included in the trace instruction information in some cases. The robot-arm control circuitry 140 performs feedback control of the robot arm 110 by using the trace instruction information and detection signals of respective sensors of the arm sensor 111 in such a manner that the ultrasonic probe 120 moves according to the trace instruction information.

As described above, the robot arm 110 can automatically move the ultrasonic probe 120 along a body surface of an object (i.e., target examinee) P according to the trace instruction information under the control of the robot-arm control circuitry 140. This operation mode is hereinafter referred to as an automatic movement mode.

Alternatively, a user can manually move the ultrasonic probe 120 under a condition where the ultrasonic probe 120 is supported by the robot arm 110. This movement mode is hereinafter referred to as a manual movement mode. In the manual movement mode, the robot arm 110 is separated from the robot-arm control circuitry 140 and moves according to an operator's manipulation of the ultrasonic probe 120. Also in this case, the arm sensor 111 including at least the position sensor and the pressure sensor mounted on the robot arm 110 continues to operate. That is, the arm sensor 111 sequentially detects parameters of the ultrasonic probe 120 such as a position, velocity, acceleration, and biological contact pressure so as to generate detection signals, and those detection signals are sequentially transmitted to the main body 200.

Aside from the automatic movement mode and the manual movement mode, a manual assistance mode may be provided. When an operator manually moves the ultrasonic probe 120 in the manual assistance mode, the robot arm 110 assists the operator in manipulating the ultrasonic probe 120 without being separated from the robot-arm control circuitry 140. In the manual assistance mode, the robot arm 110 can provide various type of assistance as follows. For instance, in the manual assistance mode, the robot arm 110 can support the weight of the ultrasonic probe 120, keep moving velocity of the ultrasonic probe 120 constant, suppress fluctuation of the ultrasonic probe 120, and keep biological contact pressure constant.

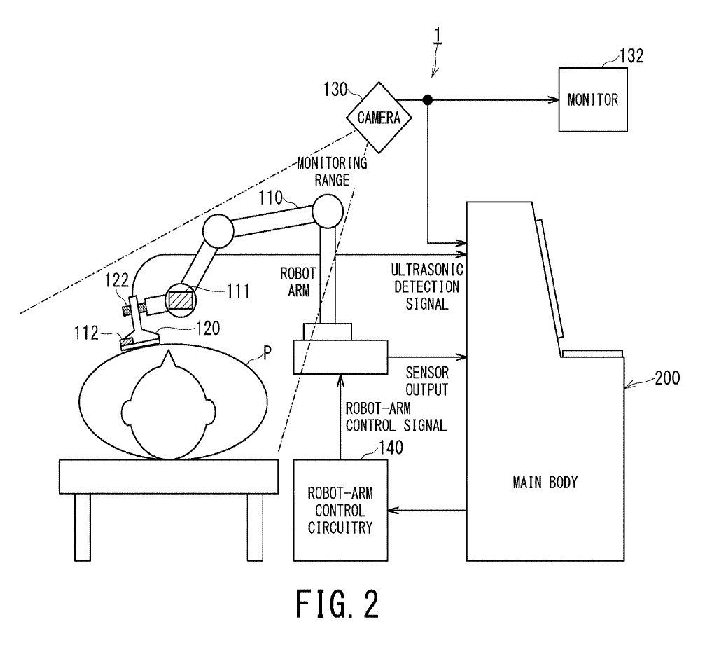

FIG. 2 is a block diagram illustrating general configuration of the ultrasonic diagnostic apparatus 1 according to the first modification of the present embodiment. The ultrasonic diagnostic apparatus 1 of the first modification further includes a camera 130 and a monitor 132 in addition to the basic configuration shown in FIG. 1. The camera 130 monitors each motion of the robot arm 110.

A position and a motion of the ultrasonic probe 120 and/or the robot arm 110 can be detected by analyzing images imaged by the camera 130. Additionally, a position of a body surface and an approximate position of an organ can be recognized by analyzing images of a living body imaged by the camera 130. The camera 130 may be configured as a visible-light camera, an infrared camera, or infrared sensor.

Images imaged by the camera 130 may be displayed on the monitor 132 disposed near the main body 200. The monitor 132 can display ultrasonic images in parallel or in switching display, in addition to images imaged by the camera 130.

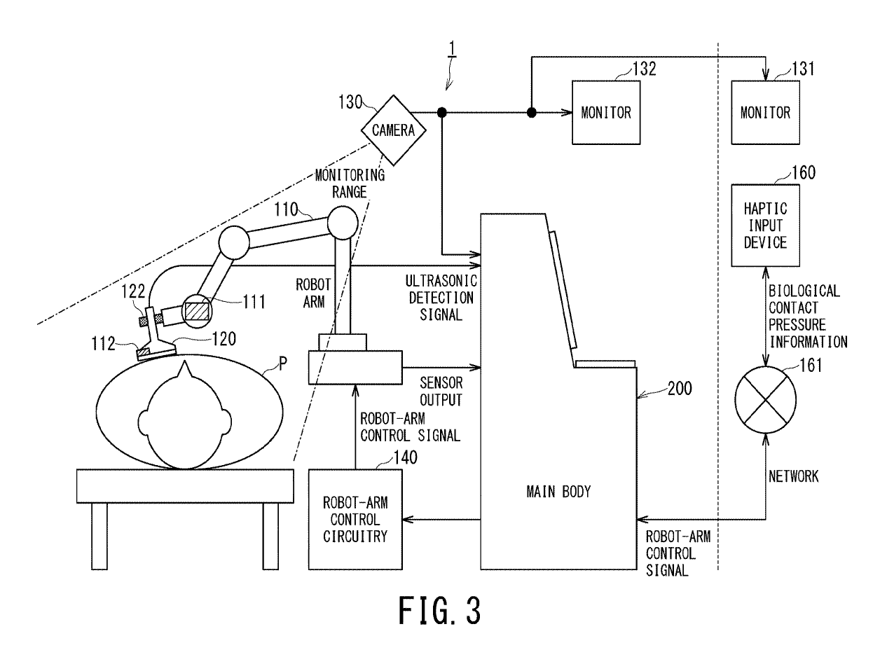

FIG. 3 is a block diagram illustrating general configuration of the ultrasonic diagnostic apparatus 1 according to the second modification of the present embodiment. The ultrasonic diagnostic apparatus 1 of the second modification further includes a haptic input device 160 and a monitor 131 in addition to the configuration of the first modification shown in FIG. 2. The haptic input device 160 and the monitor 131 are installed at, e.g., a remote place far from the main body 200. The haptic input device 160 is connected to the main body 200 and the robot-arm control circuitry 140 via the network 161 such as the internet. The haptic input device 160 is configured such that an operator can manually drive the robot arm 110 by operating the haptic input device 160 while viewing the monitor 131. The haptic input device 160 is equipped with a so-called haptic device.

The haptic input device 160 reproduces biological contact pressure of the ultrasonic probe 120 detected by the arm sensor 111 mounted on the robot arm 110. Additionally, a scanning position and a motion of the ultrasonic probe 120 on a body surface can be confirmed by watching the monitor 131. Additionally, ultrasonic images can be observed on the monitor 131 similarly to the monitor 132.

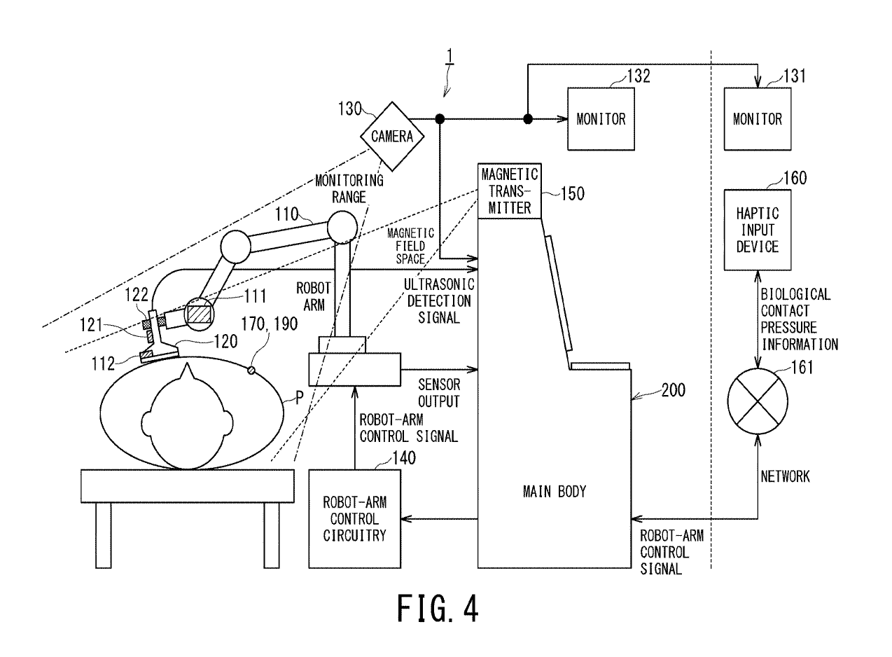

FIG. 4 is a block diagram illustrating general configuration of the ultrasonic diagnostic apparatus 1 according to the third modification of the present embodiment. The ultrasonic diagnostic apparatus 1 of the third modification further includes a position sensor configured to use a magnetic field and/or infrared rays in addition to the configuration of the second modification shown in FIG. 3. In the configuration shown in FIG. 4, the ultrasonic diagnostic apparatus 1 is further provided with position sensors such as a magnetic transmitter 150, a magnetic sensor 121, and a magnetic sensor 190.

The magnetic transmitter 150 generates a magnetic field space in a region including the ultrasonic probe 120 and the object P. The magnetic coordinate system whose origin is the magnetic transmitter 150 and the robot coordinate system can be associated with each other based on the origin and the three axes of each of those two coordinate systems.

The magnetic sensor 121 installed on the ultrasonic probe 120 provides information on a position and rotation of the ultrasonic probe 120 which is more accurate than positional information of the ultrasonic probe 120 obtained by the camera 130. As a result, the magnetic sensor 121 can enhance accuracy in positional control of the ultrasonic probe 120 performed by the robot arm 110.

The magnetic sensor 190 to be attached on a body surface of the object P detects positional information of a specific part of a living body. When positional relationship between the robot coordinate system and the biological coordinate system is changed due to a body motion, influence of the body motion can be eliminated by motion information of the object P detected by the magnetic sensor 190 attached on the body surface. Although positional information on the body surface can be detected by the camera 130, the positional information can be detected more accurately and more stably by the magnetic sensor 190.

The magnetic sensor 190 may be installed on a puncture needle. In this case, a position of a grip and/or a tip of the puncture needle can also be detected by both of the robot coordinate system and the biological coordinate system.

Additionally, the robot arm 110 can support the puncture needle on which the magnetic sensor 190 is installed. In this case, the positon of the tip of the puncture needle inside the body can be monitored and then moved or adjusted in a condition where the puncture needle is supported. Further, the tip of the puncture needle can be guided to a predetermined position inside or outside the living body.

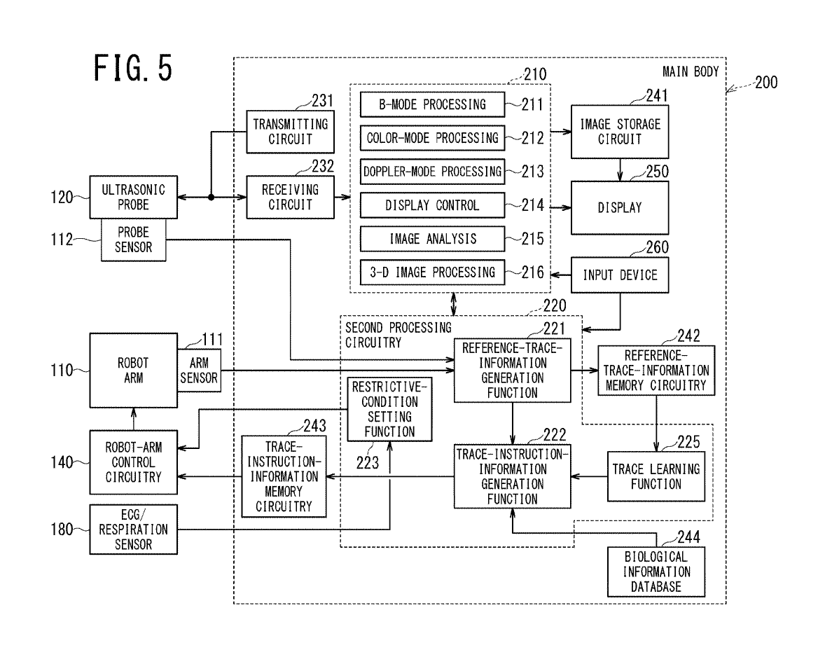

FIG. 5 is a block diagram illustrating more detailed configuration of the ultrasonic diagnostic apparatus 1 of the present embodiment, especially illustrating detailed configuration of the main body 200. The block diagram shown in FIG. 5 corresponds to the basic configuration shown in FIG. 1.

As described above, the ultrasonic probe 120, the robot arm 110, the arm sensor 111, and the robot-arm control circuitry 140 are connected to the main body 200. Aside from those components, an ECG/respiration sensor 180 can also be connected to the main body 200. Instead of the arm sensor 111 or in addition to the arm sensor 111, the ultrasonic probe 120 may be configured such that a probe sensor 112 similar to the arm sensor 111 may be mounted on the ultrasonic probe 120 as described above.

The main body 200 includes a transmitting circuit 231, a receiving circuit 232, first processing circuitry 210, a display 250, an input device 260, second processing circuitry 220, reference-trace-information memory circuitry 242, trace-instruction-information memory circuitry 243, and a biological information database 244.

The transmitting circuit 231 includes circuit components such as a trigger generation circuit, a delay circuit, and a pulsar circuit, and supplies a driving signal to the ultrasonic probe 120. The trigger generation circuit repetitively generates rate pulses at a predetermined frequency. The delay circuit delays the rate pulses by a predetermined delay amount for each transducer of the ultrasonic probe 120. The delay circuit is a circuit for focusing a transmission beam or directing a transmission beam in a desired direction. The pulsar circuit generates pulse signals based on the delayed rate pulses, and applies the pulse signals to the respective transducers of the ultrasonic probe 120.

The ultrasonic probe 120 transmits an ultrasonic signal to an object and receives the reflected ultrasonic signal from inside of the object. In addition to a one-dimensional array probe, which is generally used for an examination, a 1.25-dimensional array probe, a 1.5-dimensional array probe, a 1.75-dimensional array probe, a two-dimensional array probe capable of continuously displaying three-dimensional images, or a mechanical four-dimensional array probe capable of continuously acquiring three-dimensional data by swinging and/or rotating a one-dimensional array probe can be connected as the ultrasonic probe 120 to the main body 200. The ultrasonic signal received by the ultrasonic probe 120 is converted into an electric signal and supplied to the receiving circuit 232.

The receiving circuit 232 includes circuit components such as an amplifier circuit, an analog to digital (A/D) conversion circuit, and a beam forming circuit. In the receiving circuit 232, the amplifier circuit amplifies analog reception signals supplied from the respective transducers of the ultrasonic probe 120, and then the A/D conversion circuit converts the analog reception signals into digital signals. Afterward, the receiving circuit 232 adds a delay amount to each of the digital signals in its beam forming circuit, and then generates a reception signal corresponding to a desired beam direction by summing up those digital signals.

The first processing circuitry 210 is equipped with, e.g., a processor and a memory, and implements various types of functions by executing programs stored in the memory. The first processing circuitry 210 implements, e.g., a B-mode processing function 211, a color-mode processing function 212, a Doppler-mode processing function 213, a display control function 214, an image analysis function 215, and a three-dimensional image processing function 216.

The B-mode processing function 211 generates a B-mode image by performing predetermined processing such as envelope detection and/or logarithmic transformation on the reception signal. The color-mode processing function 212 generates a color-mode image by performing predetermined processing such as moving target indicator (MTI) filter processing and/or autocorrelation processing on the reception signal. The Doppler-mode processing function 213 generates a spectrum image by performing predetermined processing such as Fourier transform. A color-mode image, a B-mode image, and a spectrum images generated in the above-manner are stored in an image storage circuit 241 configured of components such as a Hard Disk Drive (HDD).

The display control function 214 performs display control for displaying images such as a B-mode image, a color-mode image, and a spectrum image on the display 250, and causes the display 250 to display those images and/or data related to those images.

The image analysis function 215 performs various types of image analysis on the acquired images such as a B-mode image, a color-mode image, and a spectrum image, and causes the display 250 to display the analysis result. The three-dimensional image processing function 216 three-dimensionally reconstructs B-mode beam data and/or color-mode beam data acquired together with positional information so as to generate a tomographic image in a desired direction under a multi-planar reconstruction/reformation (MPR) method and/or generate a three-dimensional image under a volume rendering (VR) method or a maximum intensity projection (MIP) method. The display 250 is a display device equipped with, e.g., a liquid crystal panel.

The input device 260 is a device for inputting various types of data and information by, e.g., an operator's manipulation. The input device 260 may be equipped with various types of information input devices such as a voice-input device and an operation device such as a keyboard, a mouse, a trackball, a joystick, and a touch panel.

The second processing circuitry 220 is equipped with, e.g., a processor and a memory, and implements various types of functions by executing programs stored in the memory similarly to the first processing circuitry 210.

The second processing circuitry 220 implements, e.g., a reference-trace-information generation function 221, a trace-instruction-information generation function 222, a restrictive-condition setting function 223, and a trace learning function 225.

The reference trace information is trace information generated on the basis of manual movement information obtained by a user's manipulation of the ultrasonic probe 120 in the state of being supported by the robot arm 110. The reference-trace-information generation function 221 is a function of acquiring the manual movement information based on the detection signals of the arm sensor 111 from a motion of the ultrasonic probe 120 operated by an operator and generating the reference trace information from the manual movement information. The generated reference trace information is stored in the reference-trace-information memory circuitry 242 configured of memories such as a HDD.

The reference trace information is information including at least a position, orientation, a moving path, and biological contact pressure of the ultrasonic probe 120. The moving path is basically defined by three-dimensional coordinate space (robot coordinate system) inside which the robot arm 110 moves. Further, in order to associate the position of the ultrasonic probe 120 with an observation target such as an organ of a living body, association information between the biological coordinate system to be set with respect to the living body and the robot coordinate system is included in the reference trace information in some cases.

A specific position of a living organ, e.g., a position of epigastrium is previously registered by the biological coordinate system, and the ultrasonic probe 120 supported by the robot arm 110 is set on the position corresponding to the registered specific position of the living organ. The position of the ultrasonic probe 120 at the time of this setting in the robot coordinate system and/or the specific position depicted in the updated ultrasonic image in the robot coordinate system are recorded. Since a specific position of a living organ is defined by both of the biological coordinate system and the robot coordinate system, the biological coordinate system and the robot coordinate system can be associated with each other. The moving path of the ultrasonic probe 120 can also be defined by the biological coordinate system.

The trace instruction information is trace information for driving the robot arm 110 so as to automatically move the ultrasonic probe 120 supported by the robot arm 110. The trace-instruction-information generation function 222 is a function of generating the trace instruction information by correcting the reference trace information generated by the reference-trace-information generation function 221 or generating the trace instruction information based on the reference trace information. The generated trace instruction information is stored in the trace-instruction-information memory circuitry 243 configured of memories such as a HDD.

The robot-arm control circuitry 140 controls driving of the robot arm 110 so as to automatically move the ultrasonic probe 120 according to the trace instruction information stored in the trace-instruction-information memory circuitry 243. The robot-arm control circuitry 140 is also equipped with, e.g., a processor and a memory, and implements various types of functions by executing programs stored in the memory, similarly to the first processing circuitry 210 and the second processing circuitry 220.

The restrictive-condition setting function 223 is a function of setting restrictive conditions for limiting each motion of the robot arm 110 in terms of, e.g., safety. The restrictive conditions are set by, e.g., an operator via the input device 260. The restrictive conditions are inputted to the robot-arm control circuitry 140 and limit a motion of the robot arm 110. For instance, when the robot arm 110 is installed beside a bed for loading object P, the space inside which the robot arm 110 can move is defined by the restrictive conditions. This is so that the robot arm 110 is prevented from colliding with, e.g., a patient, a doctor, the bed, testing equipment, a wall, or a ceiling during its operation.

The trace learning function 225 is a function of performing optimization processing on plural sets of reference trace information to generate optimized trace instruction information. The optimized trace instruction information is stored in the trace-instruction-information memory circuitry 243, and used for driving control of the robot arm 110. The optimization processing to be performed on the plural sets of reference trace information includes so-called machine-learning optimization.

The biological information database 244 is a database for storing, e.g., biological information such as a physique and an organ position of an object and image data obtained by imaging the object with other modalities such as a CT apparatus and an MRI apparatus in association with identifications of respective objects (examinees). The biological information to be stored in the biological information database 244 is used for correction processing of the trace instruction information.

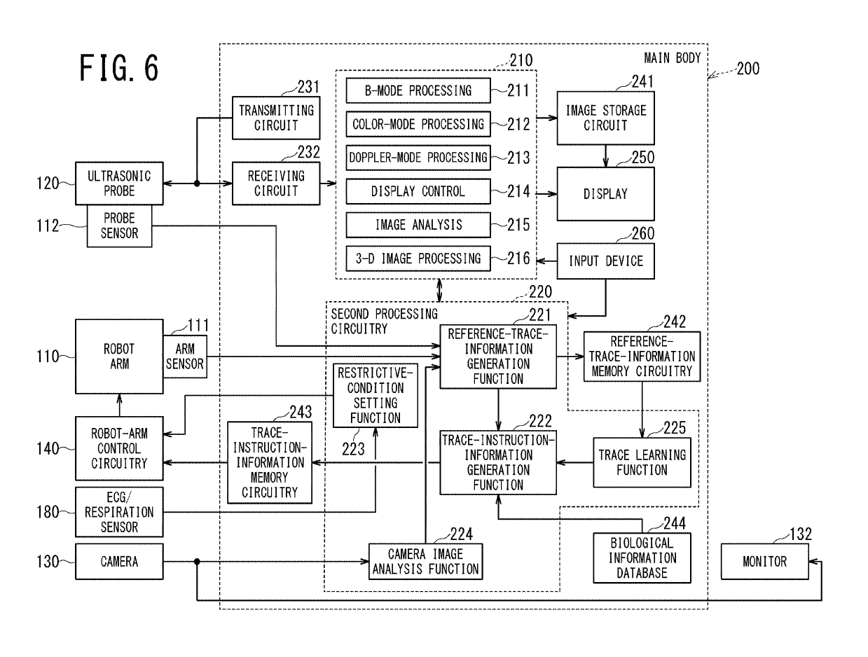

FIG. 6 is a block diagram illustrating more detailed configuration of the ultrasonic diagnostic apparatus 1 according to the first modification of the present embodiment. The block diagram shown in FIG. 6 corresponds to the configuration of the first modification shown in FIG. 2. In FIG. 6, the camera 130, the monitor 132, and a camera image analysis function 224 are added to the configuration shown in the block diagram of FIG. 5.

The camera image analysis function 224 is a function of analyzing images obtained by imaging respective motions of the robot arm 110 and the ultrasonic probe 120 with the use of the camera 130, and detecting the respective motions of the robot arm 110 and the ultrasonic probe 120 from the analysis result. A position of a body surface and an approximate position of an organ can be recognized by analyzing in-vivo images. The respective motions of the robot arm 110 and the ultrasonic probe 120, and the motion of a living body detected in the above-described manner are used for generating the reference trace information as needed.

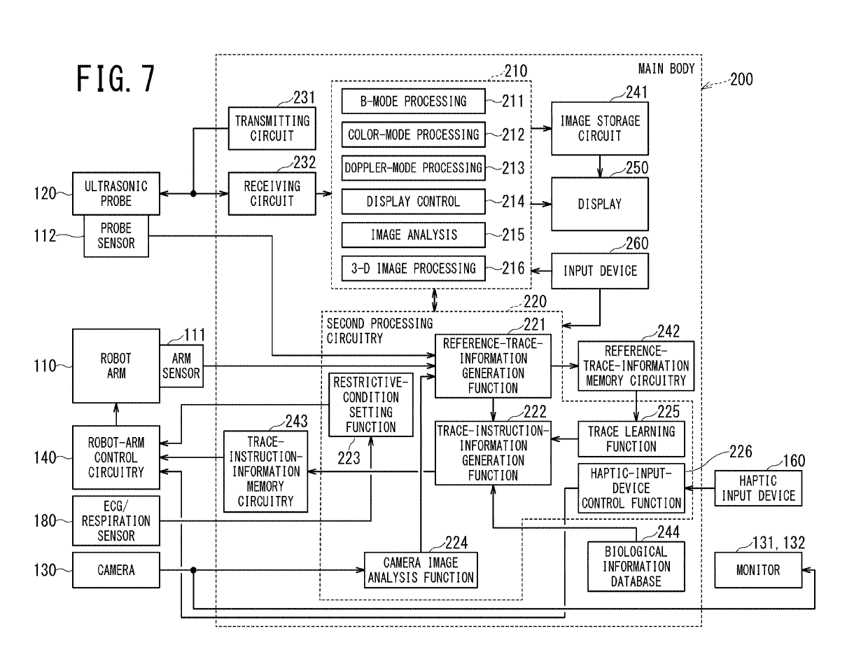

FIG. 7 is a block diagram illustrating more detailed configuration of the ultrasonic diagnostic apparatus 1 according to the second modification of the present embodiment. The block diagram shown in FIG. 7 corresponds to the configuration of the second modification shown in FIG. 3. In FIG. 7, the haptic input device 160, the monitor 131, and a haptic-input-device control function 226 are added to the configuration shown in the block diagram of FIG. 6.

The haptic-input-device control function 226 is a function of controlling the above-described haptic input device 160. The haptic-input-device control function 226 transmits biological contact pressure detected by the pressure sensor of the robot arm 110 to the haptic input device 160, and supplies the robot-arm control circuitry 140 with a control signal from the haptic input device 160 for driving the robot arm 110.

Additionally, since images imaged by the camera 130 are displayed on the monitor 131, an operator of the haptic input device 160 can watch a scanning operation of the ultrasonic probe 12 performed by the robot arm 110 at a remote place. Furthermore, a user can confirm a scanning position of the ultrasonic probe 120 on a body surface and a motion of the ultrasonic probe 120 by the monitor 131, while observing ultrasonic images.

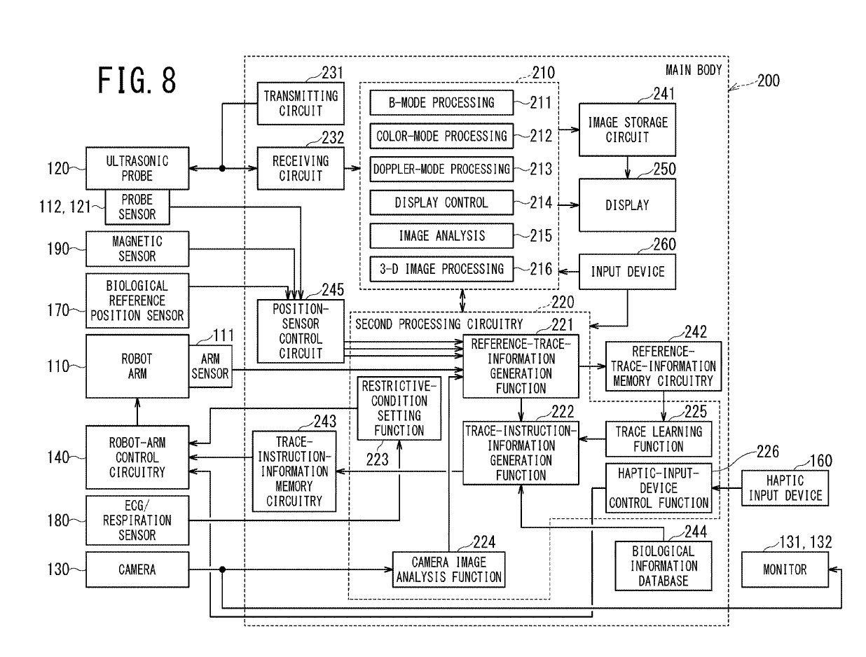

FIG. 8 is a block diagram illustrating more detailed configuration of the ultrasonic diagnostic apparatus 1 according to the third modification of the present embodiment. The block diagram shown in FIG. 8 corresponds to the configuration of the third modification shown in FIG. 4.

In the ultrasonic diagnostic apparatus 1 of the third modification, the position sensors (121, 170, 190) using a magnetic field and/or infrared rays, and a position-sensor control circuit 245 are added to the configuration of the second modification.

In the case of FIG. 8, the ultrasonic diagnostic apparatus 1 is provided with a probe sensor 121 configured as a magnetic position sensor to be mounted on the ultrasonic probe 120, and a biological reference position sensor 170 configured as a magnetic position sensor to be fixed at a predetermined reference position on a living body. The position-sensor control circuit 245 causes the probe sensor 121 and the biological reference position sensor 170 to respectively detect the positions of the sensors 121 and 170 in the magnetic coordinate system whose origin is the magnetic transmitter 150. Positional information about the probe sensor 121 and the biological reference position sensor 170 are transmitted to the reference-trace-information generation function 221 via the position-sensor control circuit 245.

The magnetic coordinate system and the robot coordinate system can be associated with each other in terms of origin and three axes. Similarly, the robot coordinate system and the biological coordinate system are associated with each other. Thus, even if positional relationship between the robot coordinate system and the biological coordinate system changes due to a body motion, influence of the body motion can be eliminated according to movement information of the biological reference position sensor 170 attached to a body surface.

Additionally, a needle position sensor 190 may be mounted as a magnetic position sensor on a puncture needle. The position of the grip and/or the needle tip of the puncture needle can be detected by the needle position sensor 190 in each of the robot coordinate system and biological coordinate system.

(Operation related to Robot Arm)

In the present embodiment and its modifications, the ultrasonic diagnostic apparatus 1 includes the robot arm 110. Hereinafter, an operation related to the robot arm 110 of the ultrasonic diagnostic apparatus 1 will be described in detail by dividing the operation into the first phase, the second phase, and the third phase.

The first phase is a phase in which an operator manually moves the ultrasonic probe 120 supported by the robot arm 110 along a body surface of an object and thereby the reference trace information is automatically generated in the ultrasonic diagnostic apparatus 1.

The second phase is a phase of generating the trace instruction information by correcting or editing the reference trace information generated in the first phase.

The third phase is a phase in which the robot arm 110 supporting the ultrasonic probe 120 is driven according to the generated trace instruction information so as to automatically move the ultrasonic probe 120 along a body surface of an object.

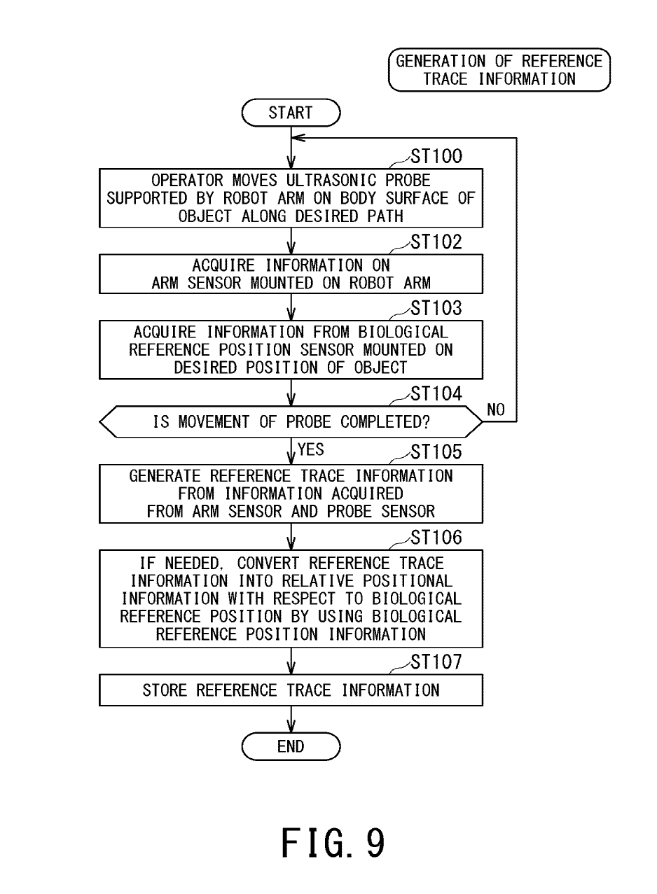

FIG. 9 is a flowchart illustrating the first case of the first phase in which the reference trace information is generated. The first case shown in FIG. 9 corresponds to the third modification (FIG. 4 and FIG. 8) in which the biological reference position sensor 170 is provided.

In the step ST100, the ultrasonic probe 120 supported by the robot arm 110 is moved along a body surface of an object so as to trace a desired path in accordance with an examination purpose.

In the step ST102, detection information of the arm sensor 111 mounted on the robot arm 110 is acquired. The arm sensor 111 is configured of the plural position sensors, the velocity sensor, and the acceleration sensor mounted on, e.g., respective joints of the robot arm 110. The arm sensor 111 acquires positional information, velocity information, and acceleration information with six degrees of freedom from those sensors. Additionally, the arm sensor 111 includes a pressure sensor, and acquires information on biological contact pressure transmitted from the ultrasonic probe 120 via the probe adapter 122. The respective information items acquired by the arm sensor 111 in the above manner are inputted to the reference-trace-information generation function 221 together with information on times at which the respective information items are acquired.

Additionally, in the step ST102, control information such as positional information of the ultrasonic probe 120 may be acquired from the probe sensors 112 and 121 mounted on the ultrasonic probe 120.

The respective information items acquired by the arm sensor 111 and/or the probe sensors 112 and 121 may be converted into the central position of the aperture of the ultrasonic probe 120 by using shape information on each of the robot arm 110 and the ultrasonic probe 120, and then may be inputted to the reference-trace-information generation function 221. Additionally, the pressure information detected by the pressure sensor may be converted into biological contact pressure at the contact area of the ultrasonic probe 120 on a body surface, and then the biological contact pressure may be inputted to the reference-trace-information generation function 221.

The positional information of the robot arm 110 detected by the arm sensor 111, and/or the positional information of the ultrasonic probe 120 detected by the probe sensors 112 and 121 can be defined as, e.g., positional information in the robot coordinate system in which a predetermined spatial position near the ultrasonic diagnostic apparatus 1 is defined as the origin and predetermined three axes perpendicularly intersecting at this origin are defined as an X axis, a Y axis, and a Z axis.

The reference trace information defined by the robot coordinate system does not depend on a relative position of an object with respect to the bed or a posture of the object.

Meanwhile, in some case, it is more convenient to define the reference trace information by the biological coordinate system which is based on a predetermined position on a body surface of an object (hereinafter, referred to as a biological reference position) and a predetermined direction (e.g., a body axis direction, in other words, a head-foot direction). In such cases, the biological reference position sensor 170 is attached to a reference position on a body surface of an object, i.e., the biological reference position. As the biological reference position, e.g., a body-surface position corresponding to a position of a xiphisternum (a protrusion which protrudes downward at the bottom end of a breastbone) may be used. The biological reference position sensor 170 is, e.g., a magnetic sensor, and detects the biological reference position by sensing a magnetic field generated by the magnetic transmitter 150 (FIG. 4). Although the number of the biological reference position sensor 170 may be one, plural biological reference position sensors 170 may be provided. For instance, one biological reference position sensor 170 may be attached to the body-surface position nearest to the xiphisternum, and another biological reference position sensor 170 may be attached to an arbitrary position on a straight line extending from the xiphisternum along the head-foot direction.

In the step ST103, detection information of the biological reference position sensor 170 is acquired. The position detected by the biological reference position sensor 170 is also defined by the robot coordinate system.

In the step ST104, whether movement of the ultrasonic probe 120 is completed or not is determined. This determination is performed on the basis of, e.g., operational information inputted from the input device 260.

In the step ST105, the reference trace information is generated from the information of the arm sensor 111 and/or the information of the probe sensors 112 and 121 acquired in the step ST102.

In the step ST106, if needed, the reference trace information is converted into relative positional information with respect to the biological reference position by using information on the biological reference position. In other words, the reference trace information defined by the robot coordinate system is converted into the reference trace information defined by the biological coordinate system.

In the step ST107, the generated reference trace information is stored in the reference-trace-information memory circuitry 242.

The processing from the steps ST102 to ST107 is performed by the second processing circuitry 221. Additionally, the processing from the steps ST102 to ST107 is not limited to the order shown in FIG. 9. For instance, information items to be detected by the respective sensors may be simultaneously acquired, and the reference trace information may be sequentially generated while the ultrasonic probe 120 is caused to move.

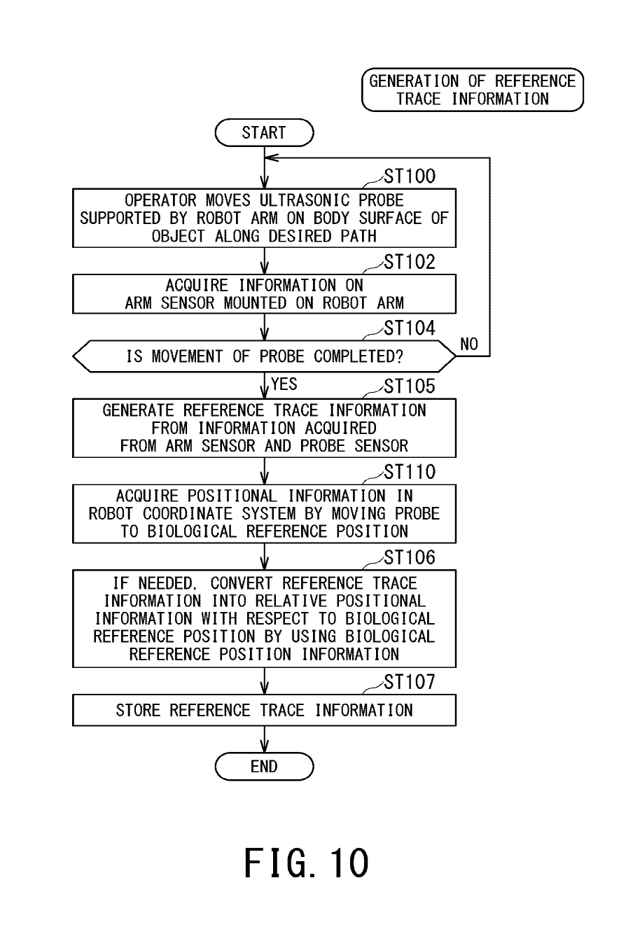

FIG. 10 is a flowchart illustrating the second case of the first phase in which the reference trace information is generated. It is not necessarily required that the biological reference position sensor 170 is used for acquiring the biological reference position information. For this reason, in the second case, the step ST110 is provided instead of the step in which the biological reference position information is acquired by using the biological reference position sensor 170 (i.e., the step ST103 in FIG. 9). The rest of the steps in FIG. 10 are the same as FIG. 9.

In the step ST110, the ultrasonic probe 120 is moved to the biological reference position, and the positional information of the biological reference position is acquired in the robot coordinate system. By placing the ultrasonic probe 120 supported by the robot arm 110 at the biological reference position (e.g., epigastrium), the probe position at that time indicated by the robot coordinate system can be converted into the biological reference position information. Further, by imaging a target region and/or a target object as an ultrasonic image and pointing the target object on the ultrasonic image, the probe position at that time indicated by the robot coordinate system can be converted into the biological reference position information.

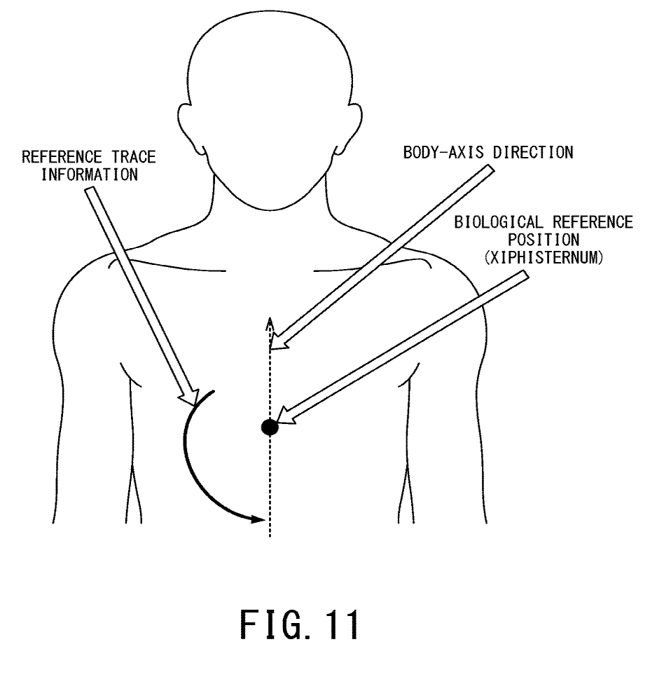

FIG. 11 is a schematic diagram illustrating a case of reference trace information and a biological reference position. In this case, the biological reference position sensor 170 is disposed at a position of a xiphisternum. When an operator moves the ultrasonic probe 120 supported by the robot arm 110, the reference trace information indicated by the bold curved arrow shown in FIG. 11 is generated.

The reference trace information includes not only time-sequential movement of each position of the ultrasonic probe 120 but also the orientation (e.g., tilt angle) of the ultrasonic probe 120 at each position and information on the biological contact pressure at each position. Additionally, the reference trace information may further include speed information and acceleration information for moving the ultrasonic probe 120.

Moreover, the reference trace information may be information converted into the biological coordinate system on the basis of an instructed structure of an examination target object and/or the body axis (i.e., head-to-hoot) direction.

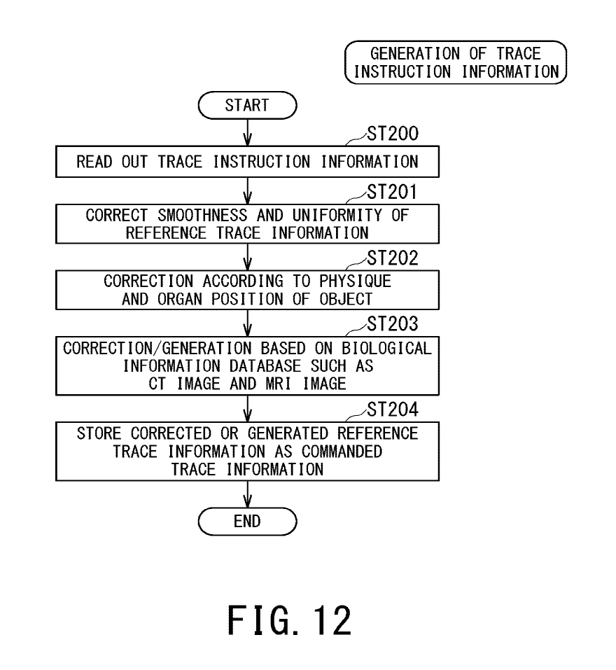

FIG. 12 is a flowchart illustrating processing of the second phase in which the trace instruction information is generated by correcting or editing the reference trace information.

In the step ST200, the reference trace information stored in the reference-trace-information memory circuitry 242 is read out.

In the step ST201, the trace instruction information with high uniformity or smoothness is generated by correcting variation and/or non-uniformity of the reference trace information. The reference trace information is generated on the basis of a trace of manually moving the ultrasonic probe 120 performed by an operator such as a medical doctor or an ultrasonic technician. Thus, no matter how skillful the operator is, the trace of movement of the ultrasonic probe 120 manipulated by the operator involves a certain degree of fluctuation or variation. For instance, even if the operator tries to keep the moving velocity of the ultrasonic probe 120 constant, the moving velocity does not become perfectly constant. Additionally, even if the operator tries to keep the orientation of the ultrasonic probe 120 constant while moving it, the orientation does not become perfectly constant. Further, vertical fluctuation with respect to a body surface is included in the trace due to hand-shaking.

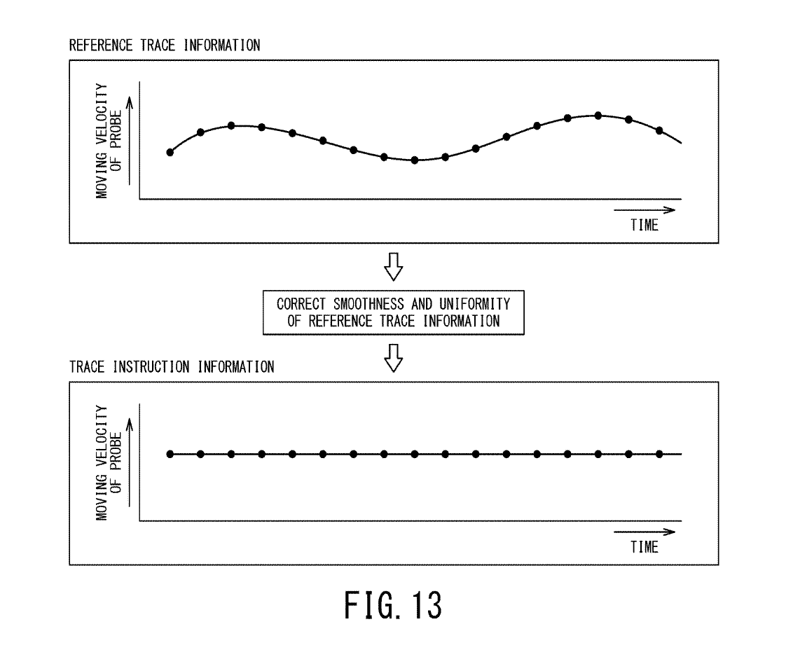

The upper part of FIG. 13 is a schematic graph illustrating a case where moving velocity of the ultrasonic probe 120 in the reference trace information is non-constant. The lower part of FIG. 13 is a schematic graph illustrating the trace instruction information corrected by the processing of the step ST201 in such a manner that moving velocity of the ultrasonic probe 120 becomes constant.

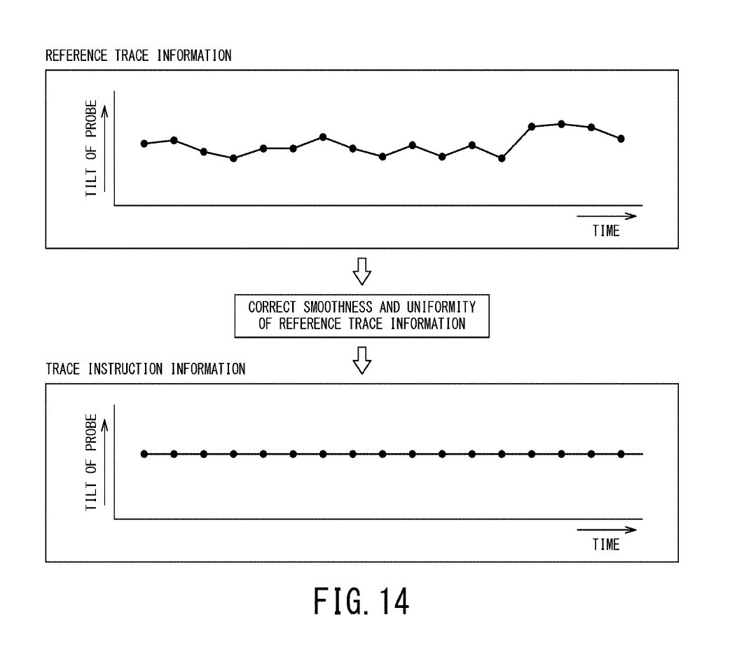

Additionally, the upper part of FIG. 14 is a schematic graph illustrating a case where a tilt of the ultrasonic probe 120 in the reference trace information is non-constant, and the lower part of FIG. 14 is a schematic graph illustrating the trace instruction information corrected by the processing of the step ST201 in such a manner that the tilt becomes constant.

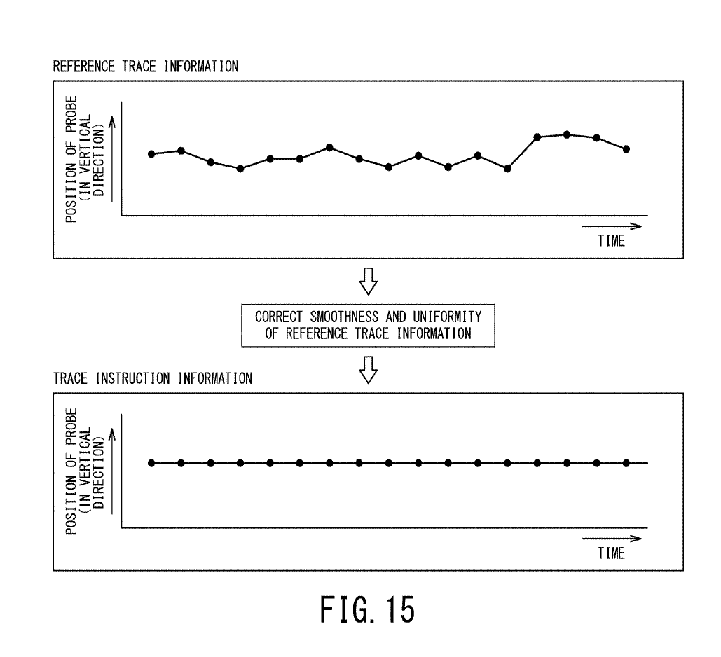

Further, the upper part of FIG. 15 is a schematic graph illustrating a case where the position of the ultrasonic probe 120 in the reference trace information is non-constant and vertically fluctuates due to hand-shaking, and the lower part of FIG. 15 is a schematic graph illustrating the trace instruction information corrected by the processing of the step ST201 in such a manner that the (vertical) position of the ultrasonic probe 120 becomes constant.

The trace instruction information can be generated as a smooth line by linearly approximating time-sequential data of the moving velocity included in the reference trace information and/or time-sequential data of the tilt of the ultrasonic probe 120 using the least square method. Additionally or alternatively, the trace instruction information can be generated as a smooth line by approximating those data by a curve of a predetermined order.

The ultrasonic probe 120 supported by the robot arm 110 is automatically moved according to the trace instruction information. In many cases, a scan of the same object (i.e., patient) using the ultrasonic probe 120 is repeated. In those cases, though the first scan is manually performed by an operator, the second and subsequent scans are automatically performed by the robot arm 110 on the basis of the trace instruction information which is generated according to the reference trace information generated in the first scan. Thus, a highly reproducible scan using a probe can be performed without imposing operational burden on an operator.

Since the trace instruction information is acquired by correcting variation and non-uniformity of the reference trace information as described above, it is possible to move the ultrasonic probe 120 under a condition where high-level uniformity which cannot realized by a skillful operator is maintained. For instance, by moving the ultrasonic probe 120 at constant velocity, cross-sectional images in parallel with each other can be imaged in such a manner that the distance between respective cross-sectional images is perfectly uniform.

An examination of the same organ (e.g., a liver) is performed on each of plural patients in some cases, and the same examination is performed on each of plural patients in e.g., a medical checkup in some cases. In the case of repeating the same examination as described above, the object (i.e., the first patient) from which the reference trace information has been acquired is different from the next object (i.e., the second patient) on which an automatic scan using the trace instruction information is to be performed. In this case, it is highly conceivable that the first patient and the second patient are significantly different in physique and organ arrangement from each other. In such a case, the trace instruction information which is generated from the reference trace information acquired from the first patient does not match the second patient in terms of organ arrangement.

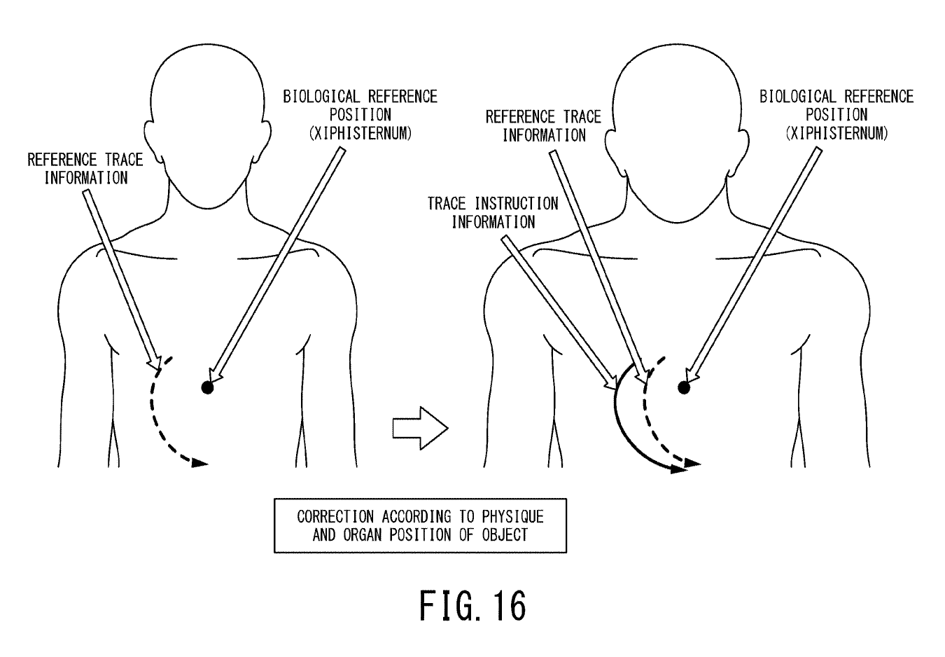

FIG. 16 illustrates a case where the object on the left side (the first patient) and the object on the right side (the second patient) are significantly different in physique from each other, and naturally, organ arrangement is different between the first patient and the second patient.

In the step ST202 of FIG. 12, the trace instruction information is further corrected in such a case according to physique and organ arrangement of each object.

For instance, organ position information in accordance with various types of physiques of patients such as weight, height, gender, and age generated from patient data such as many examination results in the past is previously stored in the biological information database 244. Then, the physique of the object (first patient) from which the reference trace information has been generated is acquired from the biological information database 244, and the organ position information associated with the physique of the object (second patient) on which an automatic scan is to be performed with the use of the robot arm 110 is also acquired from the biological information database 244. The trace instruction information can be generated by correcting the reference trace information on the basis of difference in organ position between the first patient and the second patient.

Additionally, when a diagnostic image such as a CT image or an MRI image exists for the same object (second patient) as a target of an automatic scan using the robot arm 110, the trace instruction information can be generated by more accurately correcting the reference trace information with reference to those diagnostic images. In such a case, a CT image and/or an MRI image of the object (second patient) is acquired via, e.g., a network inside a hospital and the acquired images are stored in the biological information database 244.

In the step ST203, a CT image and/or an MRI image of the object (second patient) is acquired from the biological information database 244, and the reference trace information is corrected on the basis of the acquired diagnostic images.

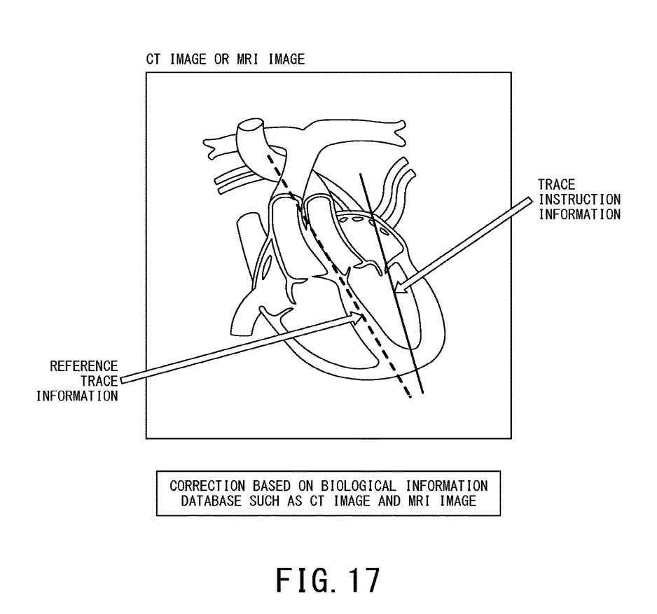

FIG. 17 is a schematic diagnostic image illustrating how the trace instruction information is generated by correcting the reference trace information based on a cardiac CT image and/or a cardiac MRI image. For instance, positioning based on nonrigid registration and an anatomical landmark (i.e., anatomical characteristic shape of a tissue of the object) is performed between CT data of respective patients. Afterward, the reference trace information is transformed according to information on organ transformation in the positioning. Additionally or alternatively, a virtual scan using a probe is performed on a three-dimensional CT image or a three-dimensional MRI image of the object (i.e., second patient). The trace of the probe in this virtual scan is generated as the reference trace information.

In the step ST204, the reference trace information generated or corrected in the above-described steps ST201 to ST203 is stored as the trace instruction information in the trace-instruction-information memory circuitry 243.

The processing from the steps ST200 to ST204 is performed by the second processing circuitry 221.

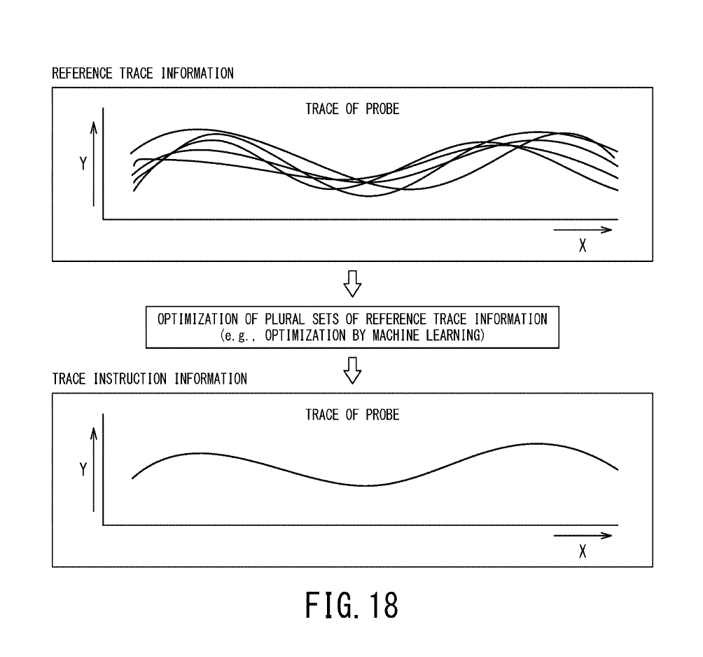

The trace instruction information can also be generated from plural sets of the reference trace information. The plural sets of the reference trace information are stored in the reference-trace-information memory circuitry 242. For instance, plural sets of the reference trace information as illustrated in the upper part of FIG. 18 are stored in the reference-trace-information memory circuitry 242.

The trace learning function 225 of the second processing circuitry 221 performs optimization processing on the plural sets of the reference trace information so as to generate one set of optimized trace instruction information as illustrated in the lower part of FIG. 18. The optimized trace instruction information is stored in the trace-instruction-information memory circuitry 243 and used for driving control of the robot arm 110.

A great amount of the reference trace information generated for the same anatomical part and/or the same disease can be acquired by plural ultrasonic diagnostic apparatuses. On the basis of such a great amount of the reference trace information and quality evaluation of the acquired images, a probe-movement trace can be optimized by using machine learning. Then, the probe-movement trace optimized by machine learning is defined as the trace instruction information, and the robot arm 110 can be driven by using this trace instruction information. Quality of the trace instruction information based on machine learning can be improved by sequentially increasing the reference trace information with time.

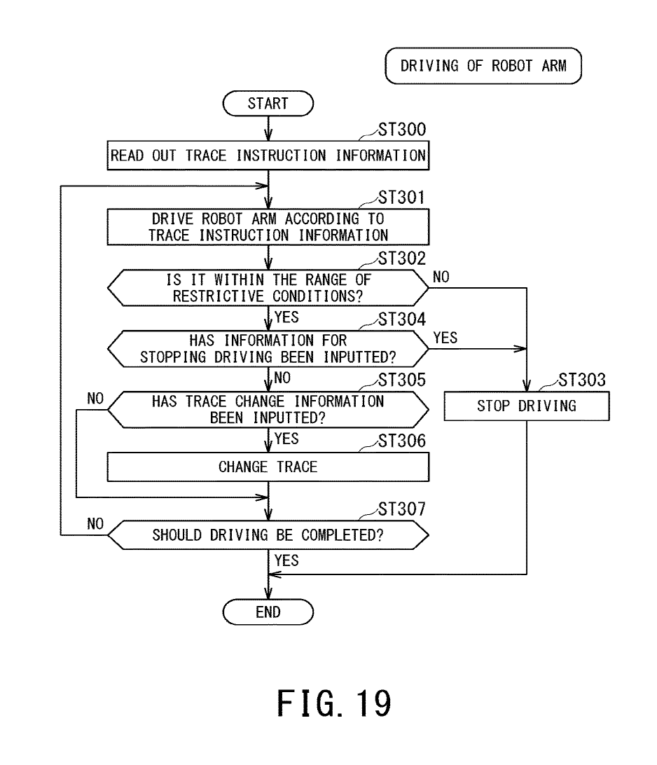

FIG. 19 is a flowchart illustrating the third phase in which the robot arm 110 is driven according to the trace instruction information stored in the trace instruction-information memory circuitry 243.

In the step ST300, the trace instruction information is read out from the trace instruction-information memory circuitry 243.

In the step ST301, the robot-arm control circuitry 140 drives the robot arm 110 according to the trace instruction information, and moves the ultrasonic probe 120 in accordance with the motion indicated by the trace instruction information. Since not only the position of the ultrasonic probe 120 but also the orientation (e.g., tilt angle) of the ultrasonic probe 120, biological contact pressure, and moving velocity are defined in the trace instruction information, the ultrasonic probe 120 automatically moves along a body surface of an object according to the trace instruction information.

The trace instruction information is generated on the basis of the reference trace information. Thus, in the case of repetitively performing the same examination on the same object, the same examination can be realized with high reproducibility without imposing operational burden on an operator. Additionally, variation and/or fluctuation of moving velocity and the tilt of the ultrasonic probe 12 attributable to manual operation are not included in the trace instruction information, and thus a probe scan more stable than that performed by a skillful operator can be achieved.

Further, since the ultrasonic probe 120 can be moved according to the trace instruction information optimized by, e.g., machine learning using plural sets of the reference trace information, more appropriate diagnosis can be achieved.

Moreover, when the object from which the reference trace information has been acquired is different from the object to be examined from now on, the ultrasonic probe 120 can be moved according to the trace instruction information matched to the organ position of the examination target object by referring to the biological information database and diagnostic images such as a CT image and an MRI image.

In the processing of driving the robot arm 110 in the step ST301, the trace instruction information may be updated by using a detection signal of the biological reference position sensor such as the magnetic sensor attached to an object. There is a possibility that a relative position of an object with respect to the bed is different for each examination. Additionally, there is a possibility that posture of an object changes during one examination. In such cases, the detection signal of the biological reference position sensor attached to an object changes from moment to moment according to the position, posture, and/or motion of the object. By sequentially updating the trace instruction information stored in the trace instruction-information memory circuitry 243 with the use of the detection signal, the ultrasonic probe 120 is caused to move in conjunction with a motion of the object on the bed. In this manner, a probe scan along the previously planned path on the body surface can be achieved. Change of the posture of the object can also be detected by analyzing time-sequential images imaged by the camera 130.

As described above, the robot arm 110 can also be driven by the haptic input device 160 disposed at a position separated from the main body 200. Information on the biological contact pressure detected by the pressure sensor mounted on the robot arm 110 is transmitted to the haptic input device 160. Thus, an operator of the haptic input device 160 can not only control motions of the ultrasonic probe 120 supported by the robot arm 110 by observing the images on the monitor 131 of the camera 130 but also control biological contact pressure by feeling the biological contact pressure of the ultrasonic probe 120.

Additionally, positions of respective organs of an object change depending on a cardiac phase (i.e., time phase of heartbeat) and a respiration phase. For this reason, an ECG/respiration sensor 180 configured to detect a cardiac phase or a respiration phase is connected to the main body 200. Then, for instance, motions of the robot arm 110 may be controlled by detecting each time phase at which positional variation of each organ due to heartbeat and respiration is small, in such a manner that the ultrasonic probe 120 is moved only in each period during which positional variation of each organ is small. Each respiration phase can also be detected by analyzing time-sequential images imaged by the camera 130.

In addition, it may be required to restrict driving of the robot arm 110 in terms of safety of an object. Additionally, it is sometimes required to restrict driving of the robot arm 110 depending on the position of the bed and arrangement of mechanical components around the main body 200. The restrictive-condition setting function 223 implements such a function. As the restrictive conditions, e.g., a driving range of the robot arm 110, the restricted range of moving velocity of the ultrasonic probe 120, and an acceptable range of biological contact pressure are included. These restrictive conditions are set via the input device 260 and stored in a predetermined memory.

In the step ST302, it is determined whether or not the position, velocity, and/or biological contact pressure of the robot arm 110 acquired from the arm sensor 111, or the trace instruction information are within the range of the above-described restrictive conditions. When it is determined as out of the range of the restrictive conditions, the processing proceeds to the step ST303 in which driving of the robot arm 110 is stopped or the robot arm 110 is moved to a safe position.

In the step ST304, it is determined whether or not information indicating a command to stop driving of the robot arm 110 is inputted during automatic movement of the ultrasonic probe 120. For instance, if there occurs such a situation, which an operator cannot predict, that the object on the bed suddenly changed the posture or largely moved, the operator contacts the robot arm 110. This contact on the robot arm 110 by the operator becomes information for stopping driving of the robot arm 110. In the step ST304, in synchronization with the above contact, it is determined that information for stopping driving of the robot arm 110 is inputted, and the processing proceeds to the step ST303 in which driving of the robot arm 110 is stopped.

Aside from the above contact, for example, voice information and/or biological information of an object (patient), information outputted from the magnetic sensor mounted on an object (patient), voice information of an operator, analysis information of images imaged by the camera 130, and analysis information of ultrasonic images can be used as information for stopping driving of the robot arm 110. When receiving those types of information in the step ST304, the robot-arm control circuitry 140 determines that information for stopping driving of the robot arm 110 is inputted, and then stops driving of the robot arm 110 in the step ST303.

In the step ST305, it is determined whether or not information of changing the moving trace of the robot arm 110 is inputted during automatic movement of the ultrasonic probe 120. For example, path information instructed through the haptic input device 160 can be used as trace change information. When the trace change information is inputted, the processing proceeds to the step ST306 in which the trace of driving the robot arm 110 is changed according to the inputted trace change information.

In the step ST307, it is determined whether driving by the robot arm 110 is completed or not. When driving by the robot arm 110 is not completed, the processing returns to the step ST301 and driving is continued.

Note that the processing from the steps ST300 to ST307 is performed by the robot-arm control circuitry 140.

(Ultrasonic Diagnosis Support Apparatus)

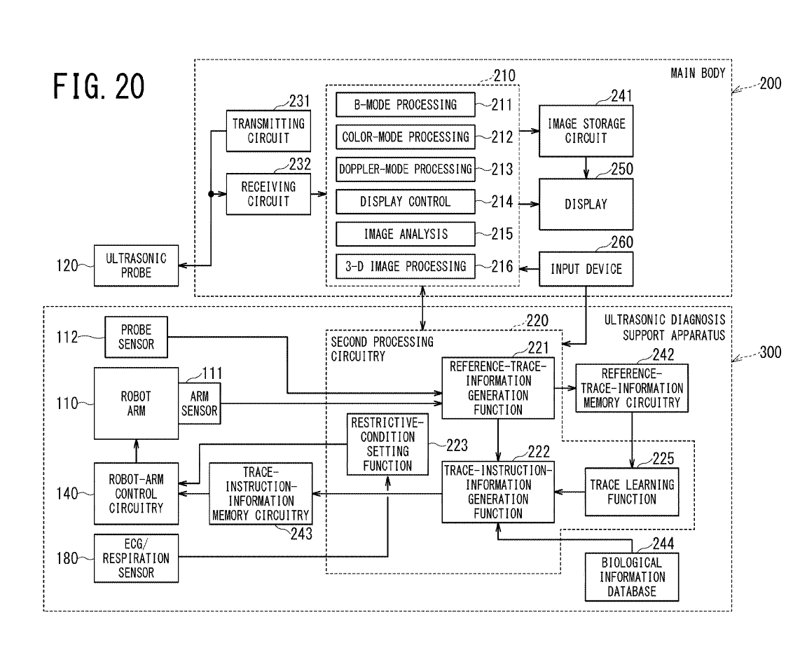

FIG. 20 is a block diagram illustrating general configuration of an ultrasonic diagnostic system of one embodiment, and the lower part of FIG. 20 corresponds to general configuration of an ultrasonic diagnosis support apparatus 300. The ultrasonic diagnosis support apparatus 300 is composed of all the components of the above-described ultrasonic diagnostic apparatus 1 excluding the configuration of the upper part of FIG. 20. That is, the ultrasonic diagnosis support apparatus 300 corresponds to all the components shown in FIG. 5 except the ultrasonic probe 120 and the main body 200 (equipped with the transmitting circuit 231, the receiving circuit 232, the first processing circuitry 210, the image storage circuit 241, the display 250, and the input device 260).

Thus, the ultrasonic diagnosis support apparatus 300 includes the robot arm 110, the robot-arm control circuit 140, the probe sensor 112, the arm sensor 111, the second processing circuitry 220, the reference-trace-information memory circuitry 242, the trace-instruction-information memory circuitry 243, a biological information database 244, and the ECG/respiration sensor 180.

As one modification, the ultrasonic diagnosis support apparatus 300 may include all the components of the first modification shown in FIG. 6 excluding the ultrasonic probe 120, the transmitting circuit 231, the receiving circuit 232, the first processing circuitry 210, the image storage circuit 241, the display 250, and the input device 260. As other two modifications, the ultrasonic diagnosis support apparatus 300 may include all the components of the second or third modification shown in FIG. 7 or FIG. 8 excluding the ultrasonic probe 120, the transmitting circuit 231, the receiving circuit 232, the first processing circuitry 210, the image storage circuit 241, the display 250, and the input device 260. Since the configuration and operations of the ultrasonic diagnosis support apparatus 300 including the above-described three modifications have been described in detail as the configuration and operations of the ultrasonic diagnostic apparatus 1, duplicate description is omitted.

By connecting the ultrasonic diagnosis support apparatus 300 as shown in FIG. 20 to a conventional ultrasonic diagnostic apparatus (i.e., the configuration of the upper part of FIG. 20), or using the ultrasonic diagnosis support apparatus 300 and a conventional ultrasonic diagnostic in combination, the above-described various types of control related to the robot arm 110 can be achieved, and thus, the ultrasonic probe 120 can be stably moved along a desired trace using the robot arm 110. Further, the conventional ultrasonic diagnostic apparatus can generate a 3-D image by acquiring three-dimensional position information of the ultrasonic image from the ultrasonic diagnosis support apparatus 300. Furthermore, the conventional ultrasonic diagnostic apparatus can display images using trace information of the probe such the reference trace information or the trace instruction information, or using positional information of the respective images. Note that the ultrasonic diagnosis support apparatus 300 is provided with an interface which transmits at least one of a position of the ultrasonic probe and a position of the ultrasonic image.

According to the ultrasonic diagnostic apparatus 1 or the ultrasonic diagnosis support apparatus 300 of the above-described embodiments as described above, the ultrasonic probe 120 can be moved along a trace more stable than a trace manually realized by an expert (e.g., at constant velocity, at a constant tilt, and at uniform interval between respective cross-sections), without depending on skills of an operator such as a medical doctor or an ultrasonic technician. Additionally, when a probe scan of the same purpose is repeated, a highly reproducible probe scan can be achieved without imposing operational burden on the operator.

Incidentally, each of the first processing circuitry 210, the second processing circuitry 220, and the robot-arm control circuitry 140 shown in FIG. 2 includes, e.g., a processor and a memory and implements predetermined functions by causing its processor to execute programs stored in the memory, as described above.

The above-described term “processor” means, e.g., a circuit such as a special-purpose or general-purpose central processing unit (CPU), an application specific integrated circuit (ASIC), a field programmable gate array (FPGA), and a programmable logic device including a simple programmable logic device (SPLD) and a complex programmable logic device (CPLD).

A processor used in each of the first processing circuitry 210, the second processing circuitry 220, and the robot-arm control circuitry 140 implements the respective functions by reading out programs stored in memory circuitry or programs directly stored in the circuit thereof and executing the programs. Each of the first processing circuitry 210, the second processing circuitry 220, and the robot-arm control circuitry 140 may be provided with one or plural processors. Additionally or alternatively, one processor may collectively execute the entire processing of at least arbitrary two or all of the first processing circuitry 210, the second processing circuitry 220, and the robot-arm control circuitry 140.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe;
a robot arm configured to support the ultrasonic probe and move the ultrasonic probe along a body surface of an object;
memory circuitry configured to store trace instruction information used by the robot arm for moving the ultrasonic probe; and
control circuitry configured to drive the robot arm in such a manner that the robot arm moves the ultrasonic probe according to the trace instruction information.

2. The ultrasonic diagnostic apparatus according to claim 1, further comprising processing circuitry configured to
generate reference trace information based on manual movement information acquired through manual movement of the ultrasonic probe supported by the robot arm, the reference trace information being used for generating the trace instruction information, and
generate the trace instruction information by correcting the reference trace information.

3. The ultrasonic diagnostic apparatus according to claim 2,
wherein the processing circuitry is configured to generate the reference trace information based on information acquired from a sensor mounted on at least one of the robot arm and the ultrasonic probe.

4. The ultrasonic diagnostic apparatus according to claim 3,
wherein the processing circuitry is configured to generate the reference trace information based on information acquired from at least one of a magnetic sensor provided to the ultrasonic probe, a gyroscope sensor provided to the ultrasonic probe, an infrared sensor provided outside the ultrasonic probe, and an image sensor provided outside the ultrasonic probe, additionally or alternatively to the sensor.

5. The ultrasonic diagnostic apparatus according to claim 2,
wherein each of the trace instruction information and the reference trace information includes probe information at each position on a moving trace of the ultrasonic probe, the probe information including at least one of a position, orientation, moving velocity, and biological contact pressure of the ultrasonic probe at each position on the moving trace; and
the processing circuitry is configured to generate the trace instruction information by correcting the probe information included in the reference trace information.

6. The ultrasonic diagnostic apparatus according to claim 2,
wherein the processing circuitry is configured to generate each of the trace instruction information and the reference trace information as information defined by relative positional information of the ultrasonic probe with respect to a reference position on a living body.

7. The ultrasonic diagnostic apparatus according to claim 2,
wherein the processing circuitry is configured to generate the trace instruction information by executing optimization processing in which plural sets of the reference trace information are used.

8. The ultrasonic diagnostic apparatus according to claim 7,
wherein the processing circuitry is configured to generate the trace instruction information as information optimized by using machine learning.

9. The ultrasonic diagnostic apparatus according to claim 2,
wherein the processing circuitry is configured to generate the trace instruction information by correcting the reference trace information based on a physique or an organ position of the object.

10. The ultrasonic diagnostic apparatus according to claim 2,
wherein the processing circuitry is configured to generate the trace instruction information by correcting the reference trace information based on a CT image or an MRI image obtained by imaging the object.

11. The ultrasonic diagnostic apparatus according to claim 2,
wherein the processing circuitry is configured to generate the trace instruction information by correcting the reference trace information based on information on a biological reference position of the object.

12. The ultrasonic diagnostic apparatus according to claim 1,
wherein the control circuitry is configured to drive the robot arm in accordance with a restrictive condition for restricting movement of the robot arm.

13. The ultrasonic diagnostic apparatus according to claim 12,
wherein the restrictive condition includes at least one of a movable range, a moving velocity range, and a biological contact pressure range of the ultrasonic probe supported by the robot arm.

14. The ultrasonic diagnostic apparatus according to claim 1,
wherein the robot arm or the ultrasonic probe is provided with (a) a position sensor, (b) a set of a position sensor and a velocity sensor, or (c) a set of a position sensor, a velocity sensor, and an acceleration sensor, for sensing movement of the ultrasonic probe; and
the control circuitry is configured to drive the robot arm based on a signal indicative of the sensed movement of the ultrasonic probe.

15. The ultrasonic diagnostic apparatus according to claim 14,
wherein the robot arm or the ultrasonic probe further includes a pressure sensor;
the control circuitry is configured to drive the robot arm further based on biological contact pressure sensed by the pressure sensor.

16. The ultrasonic diagnostic apparatus according to claim 14, further comprising at least one of an ECG sensor configured to acquire electrocardiographic information as biological information and a respiration sensor configured to acquire respiratory information as the biological information,
wherein the control circuitry is configured to drive the robot arm further based on the biological information.

17. The ultrasonic diagnostic apparatus according to claim 1, further comprising a camera configured to detect a position and motion of the ultrasonic probe or the robot arm,
wherein the control circuitry is configured to drive the robot arm based on the position and motion detected by the camera.

18. The ultrasonic diagnostic apparatus according to claim 1, further comprising a camera configured to detect a position and motion of a living body in addition to a position and motion of the ultrasonic probe or the robot arm, as position-and-motion information,
wherein the control circuitry is configured to drive the robot arm based on the position-and-motion information detected by the camera.

19. The ultrasonic diagnostic apparatus according to claim 1, further comprising a haptic input device configured to remotely detect biological contact pressure of the ultrasonic probe supported by the robot arm and remotely control driving of the robot arm,
wherein the control circuitry is configured to drive the robot arm in accordance with control of the haptic input device.

20. The ultrasonic diagnostic apparatus according to claim 1, further comprising:
a camera configure to image a position and motion of the ultrasonic probe or the robot arm;
a display configured to display the position and motion imaged by the camera; and
a haptic input device configured to remotely detect biological contact pressure of the ultrasonic probe supported by the robot arm and remotely control driving of the robot arm,
wherein the control circuitry is configured to drive the robot arm in accordance with control of the haptic input device operated while the position and motion imaged by the camera is displayed on the display.

21. The ultrasonic diagnostic apparatus according to claim 1,
wherein the control circuitry is configured to
drive the robot arm in such a manner that the ultrasonic probe is automatically moved, and
cause the robot arm to stop movement of the ultrasonic probe or change a moving path of the ultrasonic probe based on trace change information during automatic movement of the ultrasonic probe, the trace change information including at least one of (a) voice information of the object, (b) biological information of the object, (c) contact information with respect to the ultrasonic probe or the robot arm by an operator, (d) voice information of the operator, (e) information inputted from a haptic input device configured to remotely detect biological contact pressure of the ultrasonic probe supported by the robot arm, (f) analysis information of an image imaged by a camera configured to image a position and motion of the ultrasonic probe or the robot arm, and (g) positional information acquired by a position sensor attached to the object.

22. An ultrasonic diagnosis support apparatus connected to an ultrasonic diagnostic apparatus equipped with an ultrasonic probe, the ultrasonic diagnosis support apparatus comprising:
a robot arm configured to move the ultrasonic probe along a body surface of an object;
memory circuitry configured to store trace instruction information used by the robot arm for moving the ultrasonic probe; and
control circuitry configured to drive the robot arm in such a manner that the robot arm moves the ultrasonic probe according to the trace instruction information.

23. The ultrasonic diagnosis support apparatus according to claim 22, further comprising processing circuitry configured to
generate reference trace information based on manual movement information acquired through manual movement of the ultrasonic probe supported by the robot arm, the reference trace information being used for generating the trace instruction information, and
generate the trace instruction information by correcting the reference trace information.

24. The ultrasonic diagnostic support apparatus according to claim 23,
wherein the processing circuitry is configured to generate the reference trace information based on information acquired from a sensor mounted on at least one of the robot arm and the ultrasonic probe.

25. The ultrasonic diagnostic support apparatus according to claim 22, further comprising an interface transmitting at least one of a position of the ultrasonic probe and a position of an ultrasonic image to the ultrasonic diagnostic apparatus.
